(19) United States
(12) Patent Application Publication
(10) Pub. No.: US 2011/0118772 A1
(43) Pub. Date: May 19, 2011

(54) DELIVERY WIRE ASSEMBLY FOR OCCLUSIVE DEVICE DELIVERY SYSTEM

(75) Inventors: Hancun Chen, San Ramon, CA (US); Richard Murphy, Sunnyvale, CA (US); Jimmy Dao, San Jose, CA (US)

(73) Assignee: BOSTON SCIENTIFIC SCIMED, INC., Maple Grove, MN (US)

(21) Appl. No.: 12/944,254

(22) Filed: Nov. 11, 2010

Related U.S. Application Data
(60) Provisional application No. 61/261,213, filed on Nov. 13, 2009.

(51) Int. Cl. A61B 17/00

(52) U.S. Cl. 606/191

(57) ABSTRACT

A core wire of a delivery wire assembly for delivery of an occlusive device to a location in a patient's vasculature, includes a wire having a polyimide coating, with a polymer jacket disposed around the polyimide coating, and a hypotube disposed around the polymer jacket. The delivery wire assembly further comprises a delivery wire conduit including a coil disposed around the wire, wherein a proximal end of the coil is connected to a distal end of the hypotube. The polyimide coating may be enhanced with a nano-particle or micro-fiber ceramic, such as yttria-stabilized zirconia.

### RELATED APPLICATION DATA

The present application claims the benefit under 35 U.S.C. §119 to U.S. provisional patent application Ser. No. 61/261,213, filed Nov. 13, 2009. The foregoing application is hereby incorporated by reference into the present application in its entirety.

### FIELD

The field of the disclosed inventions generally relates to systems and delivery devices for implanting vaso-occlusive devices for establishing an embolus or vascular occlusion in a vessel of a human or veterinary patient. More particularly, the disclosed inventions relate to a delivery wire assembly.

### BACKGROUND

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand about a “primary” mandrel. The coil is then wrapped around a larger, “secondary” mandrel, and heat treated to impart a secondary shape. For example, U.S. Pat. No. 4,994,069, issued to Ritchart et al., describes a vaso-occlusive coil that assumes a linear, helical primary shape when stretched for placement through the lumen of a delivery catheter, and a folded, convoluted secondary shape when released from the delivery catheter and deposited in the vasculature.

In order to deliver the vaso-occlusive devices to a desired site in the vasculature, e.g., within an aneurismal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 90°, “J”, “S”, or other bending shape, depending on the particular anatomy of the patient, so that it will stay in a desired position for releasing one or more vaso-occlusive device(s) into the aneurysm once the guidewire is withdrawn. A delivery or “pusher” wire is then passed through the micro-catheter, until a vaso-occlusive device coupled to a distal end of the delivery wire is extended out of the distal end opening of the micro-catheter and into the aneurysm. The vaso-occlusive device is then released or “detached” from the end delivery wire, and the delivery wire is withdrawn back through the catheter. Depending on the particular needs of the patient, one or more additional occlusive devices may be pushed through the catheter and released at the same site.

One well-known way to release a vaso-occlusive device from the end of the pusher wire is through the use of an electrolytically severable junction, which is a small exposed section or detachment zone located along a distal end portion of the pusher wire. The detachment zone is typically made of stainless steel and is located just proximal of the vaso-occlusive device. An electrolytically severable junction is susceptible to electrolysis and disintegrates when the pusher wire is electrically charged in the presence of an ionic solution, such as blood or other bodily fluids. Thus, once the detachment zone exits out of the catheter distal end and is exposed in the vessel blood pool of the patient, a current applied through an electrical contact to the conductive pusher wire completes an electrolytic detachment circuit with a return electrode, and the detachment zone disintegrates due to electrolysis.

In “monopolar” systems, return electrodes include electrodes attached to the patient's skin and conductive needles inserted through the skin at a remote site. In “bipolar” systems, return electrodes are located on the pusher wire, e.g. on a delivery wire conduit, but electrically insulated from the conductive path ending in the detachment zone. The anode is made up of a polyimide insulated core wire, which runs through the pusher wire, is attached to the electrical contact at the proximal end, and forms the detachment zone at the distal end.

Perceived problems with current bipolar vaso-occlusive coil delivery systems include damage to the polyimide coating resulting from abrasion as the core wire is threaded through the delivery wire conduit during manufacturing. Damage to the polyimide coating in a bipolar system can lead to electrical shorts or current leakage in the electrolytic detachment system. Current leakage (a wet short) occurs when body fluid leaks into the pusher wire and makes contact with the core wire exposed by the imperfections in the insulation. An intermittent or direct hard short (a dry short) occurs when the exposed core wire makes direct contact with the inside of the pusher wire. Current leakage and electrical shorts may adversely impact detachment of the occlusive device by electrolysis.

### SUMMARY

In one embodiment of the disclosed inventions, a delivery wire assembly is provided for delivery of an occlusive device to a location in a patient's vasculature, the delivery wire assembly including a delivery wire conduit defining a conduit lumen, and a core wire disposed in the conduit lumen, wherein the core wire is at least partially covered with an abrasion resistant coating. By way of non-limiting example, the abrasion resistant coating may be a nano-particle or micro-fiber ceramic enhanced polyimide coating. By way of another, non-limiting example, the abrasion resistant coating is a nano-particle or micro-fiber ceramic layer covered with a polyimide coating enhanced with a nano-particle or a micro-fiber ceramic. In one embodiment, the abrasion resistant coating comprises yttria-stabilized zirconia. In one embodiment, the abrasion resistant coating is a polyimide layer covered with a polymer protection tube.

In another embodiment of the disclosed inventions, a delivery wire assembly is provided for delivery of an occlusive device to a location in a patient's vasculature. The assembly includes a delivery wire conduit having an inner wall defining a conduit lumen, the wall at least partially comprising an abrasion resistant surface. The assembly further includes a core wire disposed in the conduit lumen. The conduit lumen surface may have a smooth finish at least partially covered with an abrasion resistant coating. In one such embodiment, the abrasion resistant coating comprises a polyimide jacket or coating enhanced with a nano-particle or micro-fiber ceramic, such as yttria-stabilized zirconia.

In yet another embodiment of the disclosed inventions, a delivery wire assembly is provided for delivery of an occlusive device to a location in a patient's vasculature, the assembly comprising a core wire and a delivery conduit, the core wire including a wire having a polyimide coating, with a polymer jacket disposed around the polyimide coating, and a hypotube disposed around the polymer jacket. The delivery conduit includes a coil disposed around the wire, wherein a proximal end of the coil is connected to a distal end of the hypotube. The polyimide coating may be enhanced with a nano-particle or micro-fiber ceramic, such as yttria-stabilized zirconia.

### BRIEF DESCRIPTION OF THE DRAWINGS

Referring now to the drawings in which like reference numbers represent corresponding parts throughout, and in which:

FIG. 1 illustrates an occlusive coil delivery system, according to one embodiment.

FIG. 2 is a longitudinal cross-sectional view of a delivery wire assembly, according to one embodiment.

FIG. 3 illustrates an occlusive coil in a natural state mode, illustrating one exemplary secondary configuration.

FIGS. 4-6 are detailed longitudinal cross-sectional views of delivery wire assemblies according to various embodiments, each with additional details of the core wire shown in a magnified inset.

FIGS. 7-10 are detailed longitudinal cross-sectional views of delivery wire assemblies according to various embodiments, each with additional details of the delivery wire conduit shown in a magnified inset.

### DETAILED DESCRIPTION OF THE ILLUSTRATED EMBODIMENTS

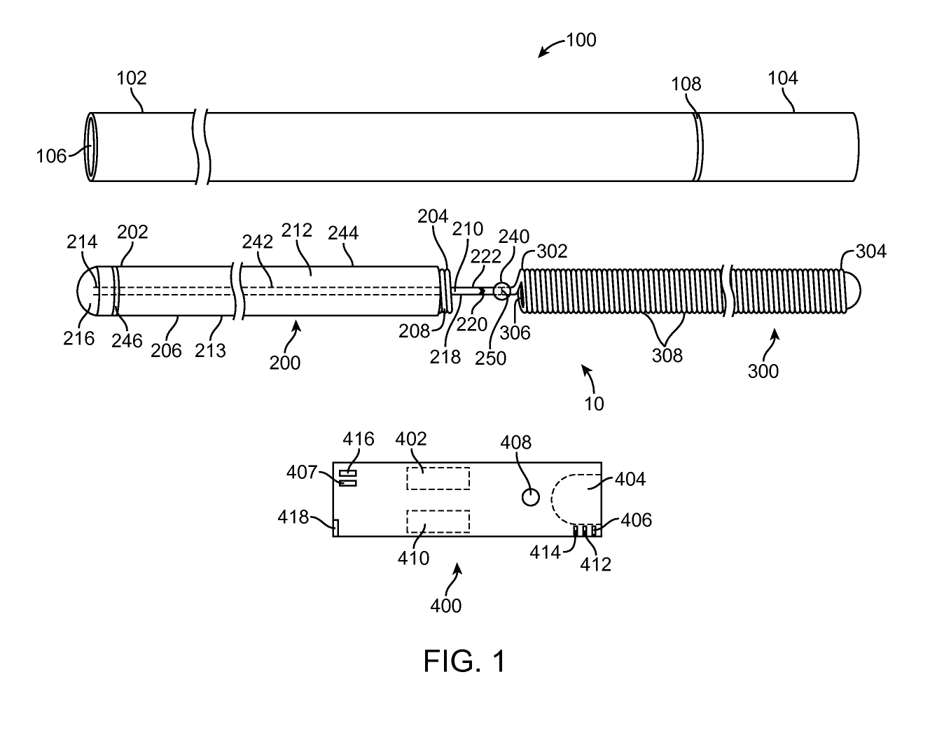

FIG. 1 illustrates an occlusive coil delivery system 10 according to one embodiment. The system 10 includes a number of subcomponents or sub-systems. These include a delivery catheter 100, a delivery wire assembly 200, an occlusive coil 300, and a power supply 400. The delivery catheter 100 includes a proximal end 102, a distal end 104, and a lumen 106 extending between the proximal and distal ends 102, 104. The lumen 106 of the delivery catheter 100 is sized to accommodate axial movement of the delivery wire assembly 200. Further, the lumen 106 is sized for the passage of a guidewire (not shown) which may optionally be used to properly guide the delivery catheter 100 to the appropriate delivery site.

The delivery catheter 100 may include a braided-shaft construction of stainless steel flat wire that is encapsulated or surrounded by a polymer coating. By way of non-limiting example, HYDROLENE® is a polymer coating that may be used to cover the exterior portion of the delivery catheter 100. Of course, the system 10 is not limited to a particular construction or type of delivery catheter 100 and other constructions known to those skilled in the art may be used for the delivery catheter 100.

The inner lumen 106 may be advantageously coated with a lubricious coating such as PTFE to reduce frictional forces between the delivery catheter 100 and the respective delivery wire assembly 200 and occlusive coil 300 being moved axially within the lumen 106. The delivery catheter 100 may include one or more optional marker bands 108 formed from a radiopaque material that can be used to identify the location of the delivery catheter 100 within the patient's vasculature system using imaging technology (e.g., fluoroscope imaging). The length of the delivery catheter 100 may vary depending on the particular application, but generally is around 150 cm in length. Of course, other lengths of the delivery catheter 100 may be used with the system 10 described herein.

The delivery catheter 100 may include a distal end 104 that is straight as illustrated in FIG. 1. Alternatively, the distal end 104 may be pre-shaped into a specific geometry or orientation. For example, the distal end 104 may be shaped into a “C” shape, an “S” shape, a “J” shape, a 45° bend, a 90° bend. The size of the lumen 106 may vary depending on the size of the respective delivery wire assembly 200 and occlusive coil 300, but generally the diameter of the lumen 106 of the delivery catheter 100 (I.D. of delivery catheter 100) is less than about 0.02 inches. The delivery catheter 100 is known to those skilled in the art as a microcatheter. While not illustrated in FIG. 1, the delivery catheter 100 may be utilized with a separate guide catheter (not shown) that aids in guiding the delivery catheter 100 to the appropriate location within the patient's vasculature.

Still referring to FIG. 1, the system 10 includes a delivery wire assembly 200 configured for axial movement within the lumen 106 of the delivery catheter 100. The delivery wire assembly 200 generally includes a proximal end 202 and a distal end 204. The delivery wire assembly 200 includes a delivery wire conduit 213, which has a proximal tubular portion 206 and a distal coil portion 208. The proximal tubular portion 206 may be formed from, for example, a flexible stainless steel hypotube. The distal coil portion 208 may be formed from, for example, stainless steel wire. The distal coil portion 208 may be joined to the proximal tubular portion 206 in an end-to-end arrangement.

The delivery wire assembly 200 further includes a core wire 210 that extends from the proximal end 202 of the delivery wire assembly 200 to a location that is distal with respect to the distal end 204 of the delivery wire assembly 200. The core wire 210 is disposed within a conduit lumen 212 that extends within an interior portion of the delivery wire conduit 213. The distal end of the conduit lumen 212 is sealed with a stopper 252. The core wire 210 is formed from an electrically conductive material such as stainless steel wire. The proximal end 214 of the core wire 210 (shown in phantom) is electrically coupled to an electrical contact 216 located at the proximal end 202 of the delivery wire assembly 200. The electrical contact 216 may be formed from a metallic solder (e.g., gold) that is configured to interface with a corresponding electrical contact (not shown) in the power supply 400. The core wire 210 is connected to the delivery wire conduit 213 as described below. The core wire 210 functions as a tether to the occlusive coil 300, such that when the delivery wire assembly 200 is pulled proximally, the occlusive coil 300 can also be withdrawn prior to coil detachment.

A portion of the core wire 210 is coated with an insulative coating 218. The entire length of the core wire 210 is coated with an insulative coating 218, except for the proximal end 214 of the core wire 210 that contacts the electrical contact 216, and a small region 220 located in a portion of the core wire 210 that extends distally with respect to the distal end 204 of the delivery wire assembly 200. This latter, “bare” portion of the core wire 210 forms the electrolytic detachment zone 220, which dissolves upon application of electrical current from the power supply 400.

In some embodiments, the insulative coating 218 is also abrasion resistant. For instance, in the embodiment in FIG. 4, the insulative and abrasion resistant coating 218 includes a polyimide layer 254 strengthened with biocompatible ceramic materials, such as yttria-stablized zirconia (“YSZ”). The ceramic materials are formed into nano-particles or micro-fibers and incorporated into the polyimide layer 254. The YSZ increases the abrasion resistance of the polyimide and the electrical insulation performance, because, in addition to hardness, YSZ also has a high dielectric constant.

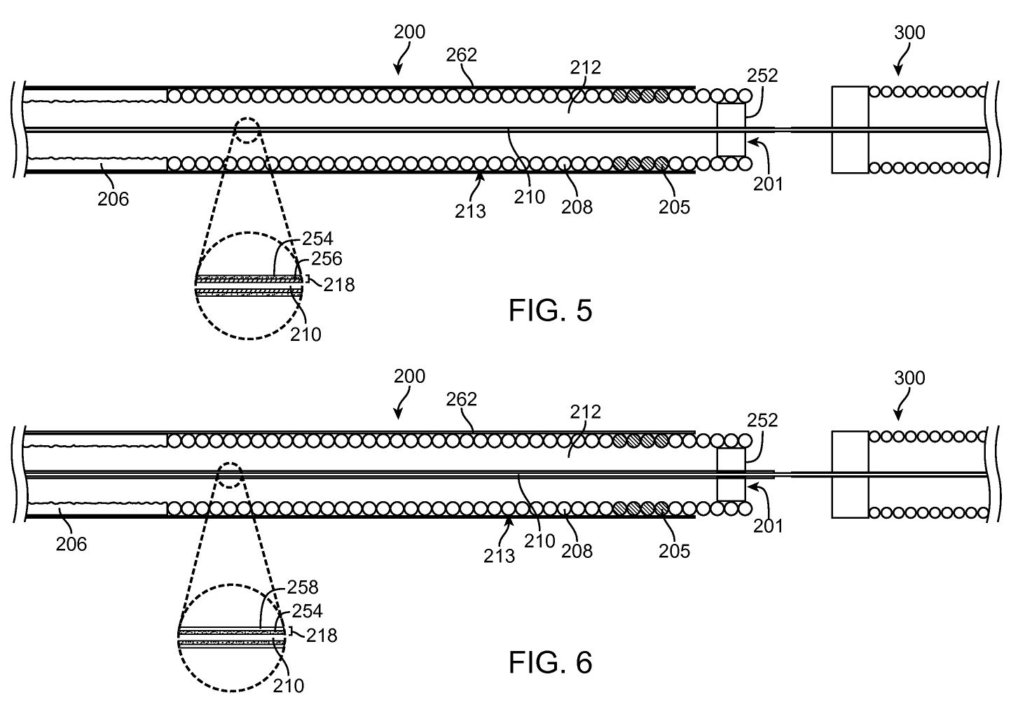

In the embodiment in FIG. 5, the insulative and abrasion resistant coating 218 includes a layer 256 of nano-particles or micro-fibers of biocompatible ceramic materials, such as YSZ is bonded to the core wire 210. Then a polyimide layer 254 is bonded on top of the ceramic layer 256. The polyimide layer 254 can also be strengthened with biocompatible ceramic materials as described above.

In the embodiment in FIG. 6, the insulative and abrasion resistant coating 218 includes a polyimide layer 254 bonded to the core wire 210. Then the polyimide layer 254 coated core wire 210 is encased in a polymer (i.e., polyimide) protection tube jacket 258. The polyimide components 254, 258 can also be strengthened with biocompatible ceramic materials as described above.

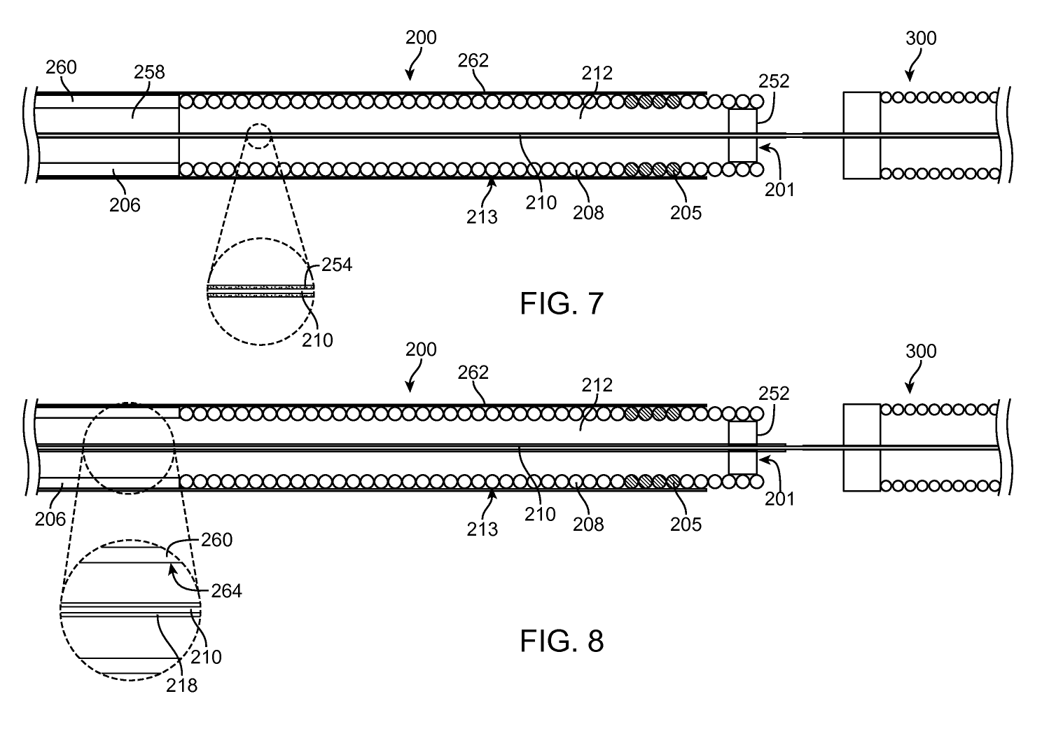

In the embodiment in FIG. 7, the insulative and abrasion resistant coating 218 in the proximal tubular portion 206 of the delivery wire conduit 213 includes a polyimide layer 254 bonded to the core wire 210. Then the polyimide layer 254 coated core wire 210 is encased in a polymer (i.e., polyimide) protection tube jacket 258 and a metal hypotube 260 in one step, using forming mills and welding lasers. The polyimide components 254, 258 can also be strengthened with biocompatible ceramic materials as described above.

In other embodiments, the inner surface 264 of the hypotube 260 in the proximal tubular portion 206 of the delivery wire conduit 213 is treated to reduce abrasion to the insulative coating 218 of the core wire 210. For instance, in the embodiment in FIG. 8, the inner surface 264 of the hypotube 260 has been treated with enhanced finishing, resulting in a smoother surface.

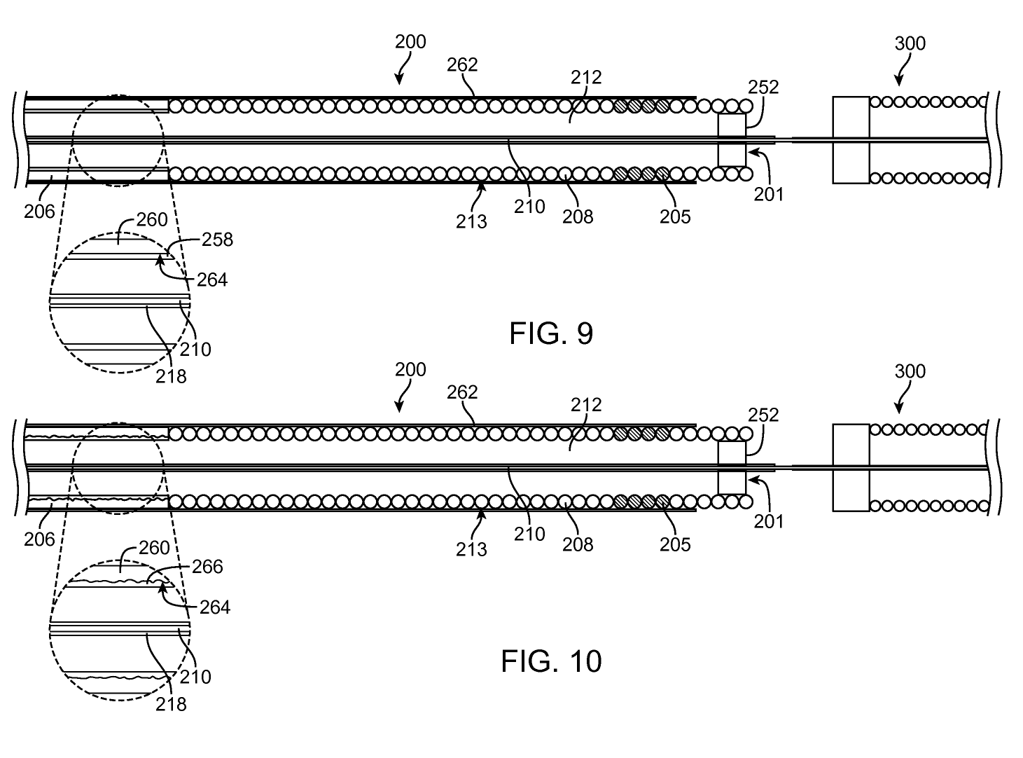

In the embodiment in FIG. 9, the inner surface 264 of the hypotube 260 is covered with a polymer (i.e., polyimide) protection tube jacket 258. The polyimide components 254, 258 can also be strengthened with biocompatible ceramic materials as described above.

In the embodiment in FIG. 10, the inner surface 264 of the hypotube 260 is covered with a polyimide coating 266. The polyimide coating 266 can also be strengthened with biocompatible ceramic materials as described above.

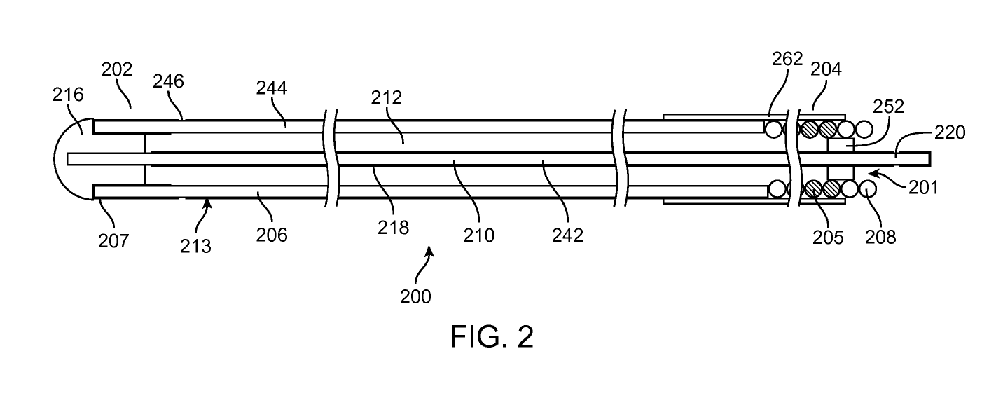

FIG. 2 illustrates a longitudinal cross-sectional view of the delivery wire assembly 200 according to one embodiment. Similar elements of this embodiment are identified with the same reference numbers as discussed above with respect to FIG. 1. The delivery wire assembly 200 includes a proximal end 202 and a distal end 204 and measures between around 184 cm to around 186 cm in length. The delivery wire assembly 200 includes a delivery wire conduit 213 with a proximal tubular portion 206, a distal coil portion 208, and a distal opening 201. The proximal tubular portion 206 may be formed from stainless steel hypotube having an outer diameter (OD) of 0.01325 inches and inner diameter (ID) of 0.0075 inches. The length of the hypotube section may be between around 140 cm to around 150 cm.

As seen in FIG. 2, a distal coil portion 208 is joined in end-to-end fashion to the distal face of the proximal tubular portion 206. The joining may be accomplished using a weld or other bond. The distal coil portion 208 may have a length of around 39 cm to around 41 cm in length. The distal coil portion 208 may comprise a coil of 0.0025 inches×0.006 inches. The first dimension generally refers to the OD of the coil wire that forms the coil. The latter dimension generally refers to the internal mandrel used to wind the coil wire around to form the plurality of coil winds and is the nominal ID of the coil.

One or more marker coils 205 of the distal coil portion 208 may be formed from a radiopaque material. For example, the distal coil portion 208 may include a segment of stainless steel coil (e.g., 3 cm in length), followed by a segment of platinum coil (which is radiopaque and also 3 mm in length), followed by a segment of stainless steel coil (e.g., 37 cm in length), and so on and so forth.

An outer sleeve 262 or jacket surrounds a portion of the proximal tubular portion 206 and a portion of the distal coil portion 208 of the delivery wire conduit 213. The outer sleeve 262 covers the interface or joint formed between the proximal tubular portion 206 and the distal coil portion 208. The outer sleeve 262 may have a length of around 50 cm to around 54 cm. The outer sleeve 262 may be formed from a polyether block amide plastic material (e.g., PEBAX 7233 lamination). The outer sleeve 262 may include a lamination of PEBAX and HYDROLENE® that may be heat laminated to the delivery wire assembly 200. The OD of the outer sleeve 262 may be less than 0.02 inches and advantageously less than 0.015 inches.

The core wire 210, which runs through the delivery wire conduit 213, terminates at electrical contact 216 at one end and extends distally with respect to the distal coil portion 208 of the delivery wire conduit 213 to the core wire distal end 222 at the other end. The core wire 210 is coated with an insulative (or insulative and abrasion resistant) coating 218 except at the electrolytic detachment zone 220 and the proximal segment coupled to the electrical contact 216. The electrolytic detachment zone 220 is located less and half a millimeter (e.g., about 0.02 mm to about 0.2 mm) distally with respect to the distal end of the distal coil portion 208. The core wire 210 may have an OD of around 0.00175 inches.

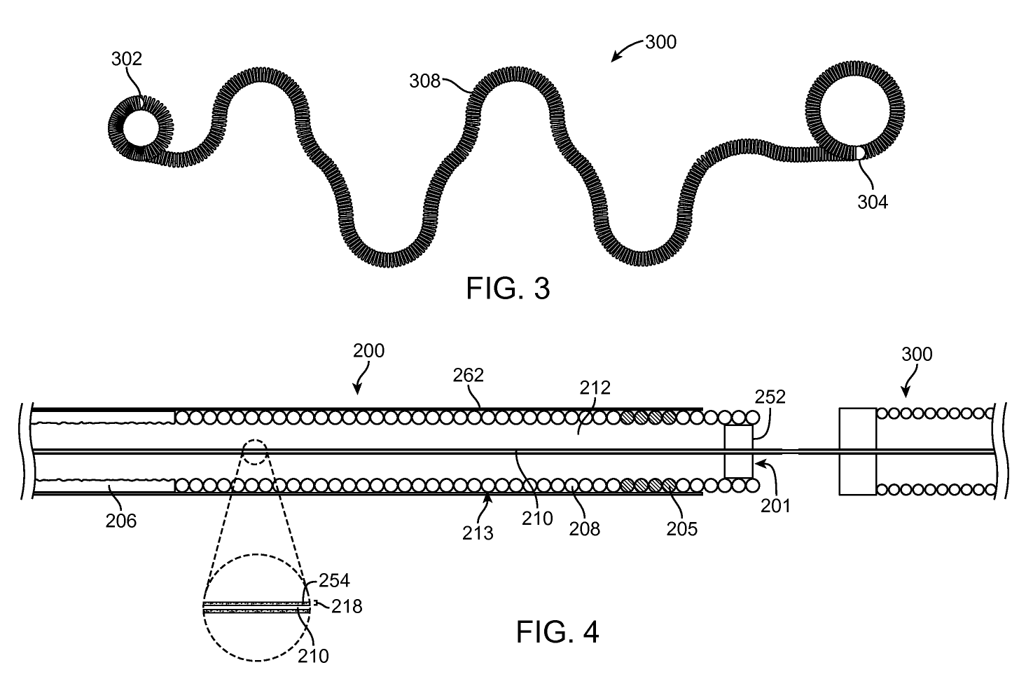

FIG. 3 illustrates one exemplary configuration of an occlusive coil 300 in a natural state. In the natural state, the occlusive coil 300 transforms from the straight configuration illustrated in, for instance, FIG. 1 into a secondary shape. The secondary shaped may include both two and three dimensional shapes of a wide variety. FIG. 3 is just one example of a secondary shape of an occlusive coil 300 and other shapes and configurations are contemplated to fall within the scope of the disclosed inventions. Also, the occlusive coil 300 may incorporate synthetic fibers over all or a portion of the occlusive coil 300 as is known in the art. These fibers may be attached directly to coil windings 308 or the fibers may be integrated into the occlusive coil 300 using a weave or braided configuration.

The occlusive coil 300 includes a proximal end 302, a distal end 304, and a lumen 306 extending there between. The occlusive coil 300 is generally made from a biocompatible metal such as platinum or a platinum alloy (e.g., platinum-tungsten alloy). The occlusive coil 300 generally includes a straight configuration (as illustrated in FIG. 1) when the occlusive coil 300 is loaded within the delivery catheter 100. Upon release, the occlusive coil 300 generally takes a secondary shape which may include three-dimensional helical configurations such as those illustrated in FIG. 3.

The occlusive coil 300 includes a plurality of coil windings 308. The coil windings 308 are generally helical about a central axis disposed along the lumen 306 of the occlusive coil 300. The occlusive coil 300 may have a closed pitch configuration as illustrated in FIG. 1. Of course, the system 10 described herein may be used with occlusive coils 300 or other occlusive structures having a variety of configurations, and is not limited to occlusive coils 300 having a certain size or configuration. Additional features or components might be used to provide mechanical interlock between the delivery wire 200 and occlusive coil 300.

The distal end 222 of the core wire 210, which includes the electrolytic detachment zone 220, is connected to the proximal end 302 of the occlusive coil 300 at a junction 250. Various techniques and devices can be used to connect the core wire 210 to the occlusive coil 300, including laser melting, and laser tack, spot, and continuous welding. It is preferable to apply an adhesive 240 to cover the junction 250 formed between the distal end 222 of the core wire 210 and the proximal end 302 of the occlusion coil 300. The adhesive 240 may include an epoxy material which is cured or hardened through the application of heat or UV radiation. For example, the adhesive 240 may include a thermally cured, two-part epoxy such as EPO-TEK® 353ND-4 available from Epoxy Technology, Inc., 14 Fortune Drive, Billerica, Mass. The adhesive 240 encapsulates the junction 250 and increases its mechanical stability.

As shown in FIG. 1, the system 10 further includes a power supply 400 for supplying direct current to the core wire 210, which contains the electrolytic detachment zone 220. In the presence of an electrically conductive fluid (including a physiological fluid such as blood, or an electrically conductive flushing solution such as saline), activation of the power supply 400 causes electrical current to flow in a circuit including the core wire electrical contact 216, the core wire 210, the electrolytic detachment zone 220, and a return electrode (not shown). After several seconds (generally less than about 10 seconds), the sacrificial electrolytic detachment zone 220 dissolves, and the occlusive coil 300 separates form the core wire 210.

The power supply 400 preferably includes an onboard energy source, such as batteries (e.g., a pair of AAA batteries), along with drive circuitry 402. The drive circuitry 402 may include one or more microcontrollers or processors configured to output a driving current. The power supply 400 illustrated in FIG. 1 includes a receptacle 404 configured to receive and mate with the proximal end 202 of the delivery wire assembly 200. Upon insertion of the proximal end 202 into the receptacle 404, the electrical contact 216 disposed on the delivery wire assembly 200 electrically couple with corresponding contacts (not shown) located in the power supply 400.

A visual indicator 406 (e.g., LED light) is used to indicate when the proximal end 202 of delivery wire assembly 200 has been properly inserted into the power supply 400. Another visual indicator 407 is activated if the onboard energy source needs to be recharged or replaced. The power supply 400 includes an activation trigger or button 408 that is depressed by the user to apply the electrical current to the sacrificial electrolytic detachment zone 220. Once the activation trigger 408 has been activated, the driver circuitry 402 automatically supplies current until detachment occurs. The drive circuitry 402 typically operates by applying a substantially constant current, e.g., around 1.5 mA.

The power supply 400 may include optional detection circuitry 410 that is configured to detect when the occlusive coil 300 has detached from the core wire 210. The detection circuitry 410 may identify detachment based upon a measured impedance value. A visual indicator 412 may indicate when the power supply 400 is supplying adequate current to the sacrificial electrolytic detachment zone 220. Another visual indicator 414 may indicate when the occlusive coil 300 has detached from the core wire 210. As an alternative to the visual indicator 414, an audible signal (e.g., beep) or even tactile signal (e.g., vibration or buzzer) may be triggered upon detachment. The detection circuitry 410 may be configured to disable the drive circuitry 402 upon sensing detachment of the occlusive coil 300.

The power supply 400 may also contain another visual indicator 416 that indicates to the operator when non-bipolar delivery wire assembly is inserted into the power supply 400. As explained in the background above, non-bipolar delivery wire assemblies use a separate return electrode that typically is in the form of a needle that was inserted into the groin area of the patient. The power supply 400 is configured to detect when a non-bipolar delivery wire assembly has been inserted. Under such situations, the visual indicator 416 (e.g., LED) is turned on and the user is advised to insert the separate return electrode (not shown in FIG. 1) into a port 418 located on the power supply 400.

Still referring to FIG. 1, the core wire 210 forms a first conductive path 242 between the electrical contact 216 and the electrolytic detachment zone 220. This first conductive path 242 may comprise the anode (+) of the electrolytic circuit when the delivery wire assembly 200 is operatively coupled to the power supply 400. A second conductive path 244, the return path, is formed by the proximal tubular portion 206 and a distal coil portion 208 of the delivery wire conduit 213. The second conductive path 244 is electrically isolated from the first conductive path 242. The second conductive path 244 may comprise the cathode (−) or ground electrode for the electrical circuit.

A ground contact 246 for the second conductive path 244 may be disposed on a proximal end of the tubular portion 206 of the delivery wire conduit 213. In one embodiment, the ground contact 246 is simply an exposed portion of the tubular portion 206 since the tubular portion 206 is part of the second conductive path 244. For instance, a proximal portion of the tubular portion 206 that is adjacent to the electrical contact 216 may be covered with an insulative coating 207 such as polyimide as illustrated in FIG. 2. An exposed region of the tubular portion 206 that does not have the insulative coating may form the ground contact 246. Alternatively, the ground contact 246 may be a ring type electrode or other contact that is formed on the exterior of the tubular portion 206.

The ground contact 246 is configured to interface with a corresponding electrical contact (not shown) in the power supply 400 when the proximal end 202 of the delivery wire assembly 200 is inserted into the power supply 400. The ground contact 246 of the second conductive path 244 is, of course, electrically isolated with respect to the electrical contact 216 of the first conductive path 242.

While various embodiments of the disclosed inventions have been shown and described, they are presented for purposes of illustration, and not limitation. Various modifications may be made to the illustrated and described embodiments (e.g., the dimensions of various parts) without departing from the scope of the disclosed inventions, which is to be limited and defined only by the following claims and their equivalents.

What is claimed is:

1. A delivery wire assembly for delivering an occlusive device to a location in a patient's vasculature, comprising:
a delivery wire conduit defining a conduit lumen; and
a core wire disposed in the conduit lumen, wherein the core wire is at least partially covered with an abrasion resistant coating.

2. The delivery wire assembly of claim 1, wherein the abrasion resistant coating is a nano-particle or micro-fiber ceramic enhanced polyimide coating.

3. The delivery wire assembly of claim 1, wherein the abrasion resistant coating is a nano-particle or micro-fiber ceramic layer covered with a polyimide coating enhanced with a nano-particle or a micro-fiber ceramic.

4. The delivery wire assembly of claim 1, wherein the abrasion resistant coating comprises yttria-stabilized zirconia.

5. The delivery wire assembly of claim 1, wherein the abrasion resistant coating is a polyimide layer covered with a polymer protection tube.

6. A delivery wire assembly for delivering an occlusive device to a location in a patient's vasculature, comprising:
a delivery wire conduit defining a conduit lumen, the conduit lumen defined by a lumen wall at least partially comprising an abrasion resistant surface; and
a core wire disposed in the conduit lumen.

7. The delivery wire assembly of claim 6, wherein the conduit lumen surface has a smooth finish.

8. The delivery wire assembly of claim 6, wherein the conduit lumen surface is at least partially covered with an abrasion resistant coating.

9. The delivery wire assembly of claim 8, wherein the abrasion resistant coating comprises a polyimide jacket or coating.

10. The delivery wire assembly of claim 9, wherein the polyimide jacket or coating is enhanced with a nano-particle or micro-fiber ceramic.

11. The delivery wire assembly of claim 10, wherein the ceramic is yttria-stabilized zirconia.

12. A delivery wire assembly for delivering an occlusive device to a location in a patient's vasculature, comprising a core wire and a delivery conduit, the core wire comprising a wire, a polyimide coating disposed around the wire, a polymer jacket disposed around the polyimide coating, and a hypotube disposed around the polymer jacket; and the delivery wire conduit comprising a coil disposed around the wire having a polyimide coating, the coil having a proximal end connected to the hypotube distal end.

13. The delivery wire assembly of claim 12, wherein the polyimide coating is enhanced with a nano-particle or micro-fiber ceramic.

14. The delivery wire assembly of claim 13, wherein the ceramic is yttria-stabilized zirconia.
